(19) United States
(12) Patent Application Publication
(10) Pub. No.: US 2024/0138774 A1
(43) Pub. Date: May 2, 2024

(54) METHOD AND APPARATUS FOR PROCESSING HUMAN BIOLOGICAL SIGNAL DATA, DEVICE, AND STORAGE MEDIUM

(71) Applicant: KINGFAR INTERNATIONAL INC., Beijing (CN)

(72) Inventors: Qichao ZHAO, Beijing (CN); Ran YANG, Beijing (CN)

(21) Appl. No.: 18/379,364

(22) Filed: Oct. 12, 2023

(30) Foreign Application Priority Data
Oct. 28, 2022 (CN) 202211337784.8

(51) Int. Cl. A61B 5/00

(52) U.S. Cl. CPC A61B 5/7275; A61B 5/7225

(57) ABSTRACT

Disclosed is a method for processing human biological signal data, including: traversing an original human biological signal with a plurality of preset peak-to-peak value adjustment time windows; acquiring an original peak-to-peak value of the original human biological signal within a previous peak-to-peak value adjustment time window; determining a target adjustment multiple of the current peak-to-peak value adjustment time window based on the original peak-to-peak value and a preset target peak-to-peak value; and determining a plurality of actual adjustment multiples of the current peak-to-peak value adjustment time window based on the target adjustment multiple, and sequentially adjusting signal values of the original human biological signal at a plurality of sampling points within the current peak-to-peak value adjustment time window based on the plurality of actual adjustment multiples.

### CROSS-REFERENCE TO RELATED APPLICATION

The present application claims the priority benefits of China patent application No. 202211337784.8, filed on Oct. 28, 2022. The entirety of China patent application No. 202211337784.8 is hereby incorporated by reference herein and made a part of this specification.

### TECHNICAL FIELD

The present application relates to the technical field of medical detection, and in particular to a method and apparatus for processing human biological signal data, a device and a storage medium.

### BACKGROUND

Human biological signals, such as photoplethysmography (PPG) and electrocardiogram (ECG) signals, are generally collected through an analog front end in a hardware circuit, and the human biological signals are amplified to a reasonable range that can be collected by an analog-to-digital converter (ADC), and then the analog signals are digitized through the ADC and read into an MCU; and the MCU performs filtering processing on the signals, such as high-pass, low-pass, band-pass and band-stop filtering to remove irrelevant noise signals, such as power frequency and aliasing signals. Human biological signals such as PPG and ECG signals can be used for calculation of signal values of heart rate (HR) and respiratory rate (RPM).

For some people, the heartbeat is relatively weak, and the measured PPG and ECG signals will also be very weak, and the waveform changes are not obvious and the waveform features are unclear, which is not conducive to the observation of PPG and ECG signals, and the signal values of heart rate (hr) and respiratory rate (rpm) cannot be accurately calculated. However, for a person who is doing sports or has just finished doing sports, his heartbeat is relatively strong, and the measured PPG and ECG signals will also be very strong, and the waveform amplitude is too large, exceeding the display range of the equipment, resulting in incomplete waveform display. Since only part of the waveform can be displayed, the waveform features cannot be clearly displayed, which is not conducive to the observation of PPG and ECG signals, and cannot accurately calculate the signal values of heart rate (hr) and respiratory rate (rpm).

In order to completely and clearly display the waveform features of PPG and ECG signals, generally, weak signals are amplified directly, or strong signals are shrunk directly. However, if the original signal is instantaneously amplified or shrunk, the change between the signals before and after processing is very abrupt, leading to the problem of waveform abnormality, etc.

### SUMMARY

In order to solve the problem of abnormality occurred in adjustment of human biological signal data and improve the accuracy of data indicators and parameters, the present application provides a method and apparatus for processing human biological signal data, a device and a storage medium.

In a first aspect, the present application provides a method for processing human biological signal data, and the following technical solution is adopted.

A method for processing human biological signal data, wherein an original human biological signal is traversed with a plurality of preset peak-to-peak value adjustment time windows; and for the original human biological signal in any one of the plurality of peak-to-peak value adjustment time windows except a first peak-to-peak value adjustment time window, the any one of the peak-to-peak value adjustment time windows is taken as a current peak-to-peak value adjustment time window, and the method includes:
when a previous peak-to-peak value adjustment time window of the current peak-to-peak value adjustment time window ends, acquiring an original peak-to-peak value of the original human biological signal within the previous peak-to-peak value adjustment time window;
determining a target adjustment multiple of the current peak-to-peak value adjustment time window based on the original peak-to-peak value and a preset target peak-to-peak value; and
determining a plurality of actual adjustment multiples of the current peak-to-peak value adjustment time window based on the target adjustment multiple, and sequentially adjusting signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the target peak-to-peak value;
wherein the plurality of actual adjustment multiples correspond to the signal values at the plurality of sampling points one by one.

Optionally, the determining a plurality of actual adjustment multiples of the current peak-to-peak value adjustment time window based on the target adjustment multiple, and sequentially adjusting signal values of the original human biological signal at a plurality of sampling points within the current peak-to-peak value adjustment time window based on the plurality of actual adjustment multiples includes:
determining an initial multiple single-step variation based on the target adjustment multiple and a preset initial actual adjustment multiple before acquiring a signal value of the original human biological signal at a first sampling point within the current peak-to-peak value adjustment time window;
determining a second actual adjustment multiple based on the initial actual adjustment multiple and the initial multiple single-step variation, and taking the second actual adjustment multiple as a current actual adjustment multiple;
adjusting the signal value at the first sampling point based on the current actual adjustment multiple; and
determining a next multiple single-step variation based on the target adjustment multiple and the current actual adjustment multiple, determining a next actual adjustment multiple based on the current actual adjustment multiple and the next multiple single-step variation, adjusting a signal value at a next sampling point based on the next actual adjustment multiple, then taking the next actual adjustment multiple as a current actual adjustment multiple, and repeating the step of determining a next multiple single-step variation based on the target adjustment multiple and the current actual adjustment multiple until at least one first preset condition is satisfied;
wherein the first preset condition includes:
the current actual adjustment multiple belongs to a first target interval, wherein the first target interval is determined by the target adjustment multiple; and
a window end time of the current peak-to-peak value adjustment time window is reached.

Optionally, if the current actual adjustment multiple belongs to the first target interval and the window end time of the current peak-to-peak value adjustment time window is not reached, signal values of the original human biological signal at the remaining sampling points within the current peak-to-peak value adjustment time window are adjusted based on the current actual adjustment multiple until the window end time of the current peak-to-peak value adjustment time window is reached.

Optionally, the plurality of actual adjustment multiples exhibit an increasing trend over time.

In a second aspect, the present application provides a method for processing human biological signal data, and the following technical solution is adopted.

A method for processing human biological signal data, wherein an original human biological signal is traversed with a plurality of preset baseline adjustment time windows; and for the original human biological signal in any one of the plurality of baseline adjustment time windows except a first baseline adjustment time window, the any one of the baseline adjustment time windows is taken as a current baseline adjustment time window, and the method includes:
when a previous baseline adjustment time window of the current baseline adjustment time window ends, acquiring an original baseline value of the original human biological signal within the previous baseline adjustment time window;
determining a target offset of the current baseline adjustment time window based on the original baseline value and a preset target baseline value; and
determining a plurality of actual offsets of the current baseline adjustment time window based on the target offset, and sequentially offsetting a baseline of the original human biological signal at a plurality of sampling points within the current baseline adjustment time window based on the plurality of actual offsets, so that the baseline of the original human biological signal within the current baseline adjustment time window gradually approaches the target baseline value;
wherein the plurality of actual offsets correspond to the baseline at the plurality of sampling points one by one.

Optionally, the determining a plurality of actual offsets of the current baseline adjustment time window based on the target offset, and sequentially offsetting a baseline of the original human biological signal at a plurality of sampling points within the current baseline adjustment time window based on the plurality of actual offsets includes:
determining an initial baseline single-step variation based on the target baseline value and a preset initial actual offset before acquiring the baseline of the original human biological signal at a first sampling point within the current baseline adjustment time window;
determining a second actual offset based on the initial actual offset and the initial baseline single-step variation, and taking the second actual offset as a current actual offset;
offsetting the baseline at the first sampling point based on the current actual offset; and
determining a next baseline single-step variation based on the target offset and the current actual offset, determining a next actual offset based on the current actual offset and the next baseline single-step variation, offsetting the baseline at a next sampling point based on the next actual offset, then taking the next actual offset as a current actual offset, and repeating the step of determining a next baseline single-step variation based on the target offset and the current actual offset until at least one second preset condition is satisfied;
wherein the second preset condition includes:
the current actual offset belongs to a second target interval, wherein the second target interval is determined by the target offset; and
a window end time of the current baseline adjustment time window is reached.

If the current actual offset belongs to the second target interval and the window end time of the current baseline adjustment time window is not reached, the baseline of the original human biological signal at the remaining sampling points within the current baseline adjustment time window is offset based on the current actual offset until the window end time of the current baseline adjustment time window is reached.

In a third aspect, the present application provides an apparatus for processing human biological signal data, and the following technical solution is adopted.

An apparatus for processing human biological signal data, wherein:
an original human biological signal is traversed with a plurality of preset peak-to-peak value adjustment time windows; and for the original human biological signal in any one of the plurality of peak-to-peak value adjustment time windows except a first peak-to-peak value adjustment time window, the any one of the peak-to-peak value adjustment time windows is taken as a current peak-to-peak value adjustment time window, and the apparatus includes:
a first acquisition module configured to, when a previous peak-to-peak value adjustment time window of the current peak-to-peak value adjustment time window ends, acquire an original peak-to-peak value of the original human biological signal within the previous peak-to-peak value adjustment time window;
a first determination module configured to determine a target adjustment multiple of a current peak-to-peak value adjustment time window based on the original peak-to-peak value and a preset target peak-to-peak value; and
an adjustment module configured to determine a plurality of actual adjustment multiples of the current peak-to-peak value adjustment time window based on the target adjustment multiples, and sequentially adjusting signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the target peak-to-peak value; wherein the plurality of actual adjustment multiples correspond to the signal values at the plurality of sampling points one by one.

In a fourth aspect, the present application provides an apparatus for processing human biological signal data, and the following technical solution is adopted.

An apparatus for processing human biological signal data, wherein an original human biological signal is traversed with a plurality of preset baseline adjustment time windows; and for the original human biological signal in any one of the plurality of baseline adjustment time windows except a first baseline adjustment time window, the any one of the baseline adjustment time windows is taken as a current baseline adjustment time window, and the apparatus includes:
a second acquisition module configured to, when a previous baseline adjustment time window of the current baseline adjustment time window ends, acquire an original baseline value of the original human biological signal within the previous baseline adjustment time window;
a second determination module configured to determine a target offset of the current baseline adjustment time window based on the original baseline value and a preset target baseline value; and
an offsetting module configured to determine a plurality of actual offsets of the current baseline adjustment time window based on the target offset, and sequentially offsetting a baseline of the original human biological signal at a plurality of sampling points within the current baseline adjustment time window based on the plurality of actual offsets, so that the baseline of the original human biological signal within the current baseline adjustment time window gradually approaches the target baseline value; wherein the plurality of actual offsets correspond to the baseline at the plurality of sampling points one by one.

In a fifth aspect, the present application provides an electronic device, and the following technical solution is adopted.

An electronic device includes a memory and a processor; wherein the memory has stored thereon a computer program to be loaded by the processor to perform the method for any item of the first aspect.

In a sixth aspect, the present application provides a computer-readable storage medium, and the following technical solution is adopted.

A computer readable storage medium has stored thereon a computer program to be loaded by a processor to perform the method for any item of the first or second aspects.

By using the above-mentioned technical solution, the peak-to-peak value of the original human biological signal, which is weak or too strong, can be gradually adjusted to a suitable degree with the display area of the waveform display device, and the baseline of the original human biological signal can also be gradually adjusted to a suitable position in the display area of the waveform display device to clearly and completely display the waveform features of the human biological signal, and the situation of waveform distortion of the human biological signal caused by directly adjusting the peak-to-peak value and the baseline can be improved.

### BRIEF DESCRIPTION OF THE DRAWINGS

FIG. 1 is a schematic diagram of traversing an original human biological signal with a plurality of peak-to-peak value adjustment time windows according to an embodiment of the present application.

FIG. 2 is a schematic flowchart of a method for processing human biological signal data according to an embodiment of the present application.

FIG. 3 is a schematic flowchart of sub-steps of Step S103 in a method for processing human biological signal data according to an embodiment of the present application.

FIG. 4 is a waveform diagram for processing a weak original human biological signal using a method for processing human biological signal data according to an embodiment of the present application.

FIG. 5 is a waveform diagram for processing an excessively strong original human biological signal using a method for processing human biological signal data according to an embodiment of the present application.

FIG. 6 is a schematic flowchart of another method for processing human biological signal data according to an embodiment of the present application.

FIG. 7 is a flowchart showing sub-steps of Step S203 in another method for processing human biological signal data according to an embodiment of the present application.

FIG. 8 is a waveform diagram for adjusting a baseline of an original human biological signal using a method for processing human biological signal data according to an embodiment of the present application.

FIG. 9 is a block diagram of an apparatus for processing human biological signal data according to an embodiment of the present application.

FIG. 10 is a block diagram of another apparatus for processing human biological signal data according to an embodiment of the present application.

FIG. 11 is a block diagram of an electronic device according to an embodiment of the present application.

### DETAILED DESCRIPTION

In order that the objects, aspects and advantages of the embodiments of the present invention will become more apparent, a more complete description of the embodiments of the present invention will be rendered by reference to the appended drawings.

In order to solve the problem of waveform distortion caused by the instantaneous amplification or reduction of a signal, the embodiment of the present application needs to realize the step-by-step amplification or reduction of an original human biological signal; therefore, a plurality of peak-to-peak value adjustment time windows are used to perform traversal sampling on the original human biological signal, i.e., the original human biological signal is segmented according to time, and the original human biological signal within each peak-to-peak value adjustment time window is processed successively.

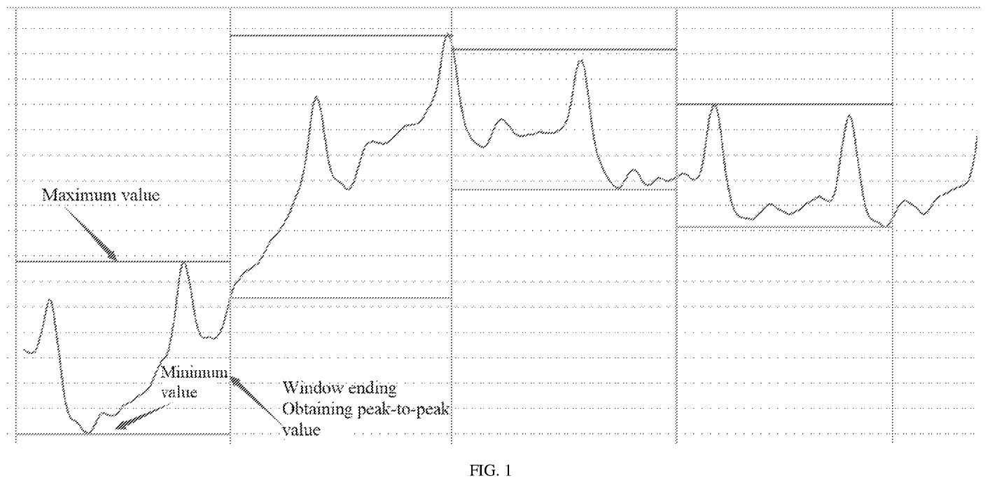

As shown in FIG. 1, for two adjacent peak-to-peak value adjustment time windows, the end time of the previous peak-to-peak value adjustment time window is the start time of the next peak-to-peak value adjustment time window. At the end of each peak-to-peak value adjustment time window, the maximum value and minimum value of the original human biological signal within the peak-to-peak value adjustment time window would be calculated, stored into a buffer with fixed length, performed sliding median filtering on the maximum value and minimum value stored in the buffer, the window mark is cleared, and the next peak-to-peak value adjustment time window is ready.

In the embodiments of the present application, the step-by-step adjustment of the signal by each peak-to-peak value adjustment time window is performed on the basis of the peak-to-peak value of the signal of the previous peak-to-peak value adjustment time window; therefore, this embodiment abandons the signal processing of the first peak-to-peak value adjustment time window, and the first peak-to-peak value adjustment time window merely provides a peak-to-peak value for the second peak-to-peak value adjustment time window. In addition, the waveform processing method for the original human biological signal of any peak-to-peak value adjustment time window except the first peak-to-peak value adjustment time window is the same.

The waveform processing method for the original human biological signal of any peak-to-peak value adjustment time window except the first peak-to-peak value adjustment time window is specifically described below, wherein any peak-to-peak value adjustment time window is used as the current peak-to-peak value adjustment time window.

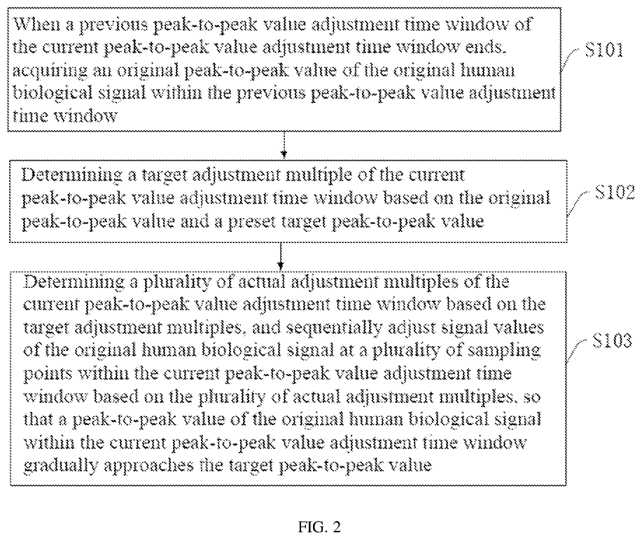

FIG. 2 is a flowchart of a method for processing human biological signal data. As shown in FIG. 2, the main flow of the method is described as follows (steps S101 to S103):

Step S101: When a previous peak-to-peak value adjustment time window of the current peak-to-peak value adjustment time window ends, acquire an original peak-to-peak value of the original human biological signal within the previous peak-to-peak value adjustment time window;

in the Step S101, the original peak-to-peak value is the difference between the maximum value and the minimum value of the original human biological signal within the previous peak-to-peak value adjustment time window. Therefore, in order to be able to acquire the original peak-to-peak value, it is necessary to ensure that the peak-to-peak value adjustment time window can find a complete signal period.

Generally, the human heartbeat frequency cannot be less than 30 times per second, and a period of human biological signals such as PPG and ECG is just a heartbeat period, so the period of the original human biological signal must not be greater than 60/30=2 s; therefore, the duration of the peak-to-peak value adjustment time window can be set to not less than 2 s, so that a complete signal period must exist within the peak-to-peak value adjustment time window, and the maximum value and minimum value of the original human biological signal can be acquired.

Step S102: Determine a target adjustment multiple of a current peak-to-peak value adjustment time window based on the original peak-to-peak value and a preset target peak-to-peak value;

in the Step S102, the target peak-to-peak value is generally determined by the longitudinal range of the display window of the waveform display device, i.e., the target peak-to-peak value is the difference between the maximum measurement value and the minimum measurement value of the longitudinal range. Of course, the target peak-to-peak value may also be smaller than the longitudinal range of the display window, to which this embodiment is not particularly limited.

The target adjustment multiple is a ratio of the target peak-to-peak value to the original peak-to-peak value. If the original peak-to-peak value is less than the target peak-to-peak value, i.e., the target adjustment multiple is greater than 1, then the signal value within the current peak-to-peak value adjustment time window needs to be amplified; if the original peak-to-peak value is greater than the target peak-to-peak value, i.e., the target adjustment multiple is greater than 0 and less than 1, then the signal value within the current peak-to-peak value adjustment time window needs to be reduced; if the original peak-to-peak value is equal to the target peak-to-peak value, i.e. the target adjustment multiple is equal to 1, there is no need to process the signal value within the current peak-to-peak value adjustment time window.

Step S103: Determine a plurality of actual adjustment multiples of the current peak-to-peak value adjustment time window based on the target adjustment multiple, and sequentially adjust signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the target peak-to-peak value.

In order to make the peak-to-peak value of the original human biological signal in the current peak-to-peak value adjustment time window gradually approach the target peak-to-peak value, a plurality of actual adjustment multiples can be determined according to the number of sampling points in the current peak-to-peak value adjustment time window, so that the plurality of actual adjustment multiples correspond to the signal values at the plurality of sampling points one by one, that is to say, when the sampling time corresponding to each sampling point in the current peak-to-peak value adjustment time window is reached, the original signal value of the sampling point is amplified or reduced according to the actual adjustment multiples corresponding to the sampling point.

In this embodiment, for the previous sampling point in the current peak-to-peak value adjustment time window, the corresponding actual adjustment multiple should not be greater than the target adjustment multiple, and the overall change trend of the actual adjustment multiple should gradually approach the target adjustment multiple, for example: the plurality of actual adjustment multiples may exhibit an increasing trend over time. Of course, it may occur that the actual adjustment times corresponding to certain sampling points fluctuate up and down.

Whether the actual adjustment multiple corresponding to the previous sampling point in the current peak-to-peak value adjustment time window reaches the target adjustment multiple or not, as long as the current peak-to-peak value adjustment time window ends, it is necessary to acquire the original peak-to-peak value according to the original human biological signal in the current peak-to-peak value adjustment time window, determine the target adjustment multiple of the next time window according to the original peak-to-peak value and the target peak-to-peak value, and continue the signal value adjustment in the next time window according to the new target adjustment multiple. That is to say, for different sampling points of the same peak-to-peak value adjustment time window, the target adjustment multiple thereof is constant; however, for the sampling points of different peak-to-peak value adjustment time windows, the target adjustment multiple thereof is generally changed, because the original peak-to-peak values may be different, but if the waveform of the original human biological signal is relatively stable, the target adjustment multiple of each peak-to-peak value adjustment time window may also be almost unchanged.

If the actual adjustment multiple reaches the target adjustment multiple when the sampling time of the previous sampling point in the current peak-to-peak adjusted time window is not reached, the actual adjustment multiple corresponding to the subsequent sampling points is set as the target adjustment multiple until the end of the current peak-to-peak value adjustment time window.

In this embodiment, the sampling time of the previous sampling point may be the end time of the current peak-to-peak value adjustment time window, or may be a time before the end of the current peak-to-peak value adjustment time window.

It should be noted that, considering the error factor, when the actual adjustment multiple belongs to the error range of the target adjustment multiple, it can be determined that the actual adjustment multiple reaches the target adjustment multiple. For example: if the target adjustment multiple is 4 and the error range thereof is ±0.1, then when the actual adjustment multiple belongs to [3.9, 4.0], it is determined that the actual adjustment multiple reaches the target adjustment multiple.

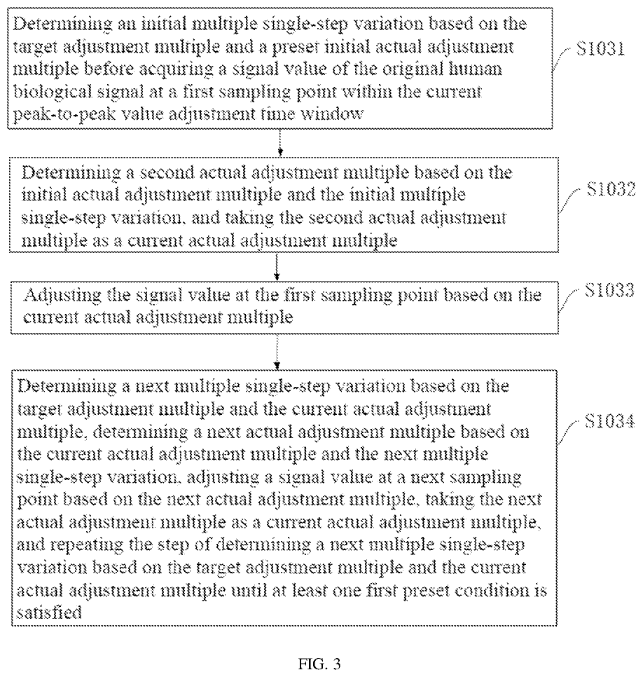

As an example to this embodiment, each actual adjustment multiple can be set by setting a single-step variation of the multiple, so that the actual adjustment multiple gradually approaches the target adjustment multiple, thereby achieving that the peak-to-peak value of the original human biological signal within the current peak-to-peak value adjustment time window gradually approaches the target peak-to-peak value. As shown in FIG. 3, the specific steps of Step S103 are as follows:

Step S1031: Determine an initial multiple single-step variation based on the target adjustment multiple and a preset initial actual adjustment multiple before acquiring a signal value of the original human biological signal at a first sampling point within the current peak-to-peak value adjustment time window;

Step S1032: Determine a second actual adjustment multiple based on the initial actual adjustment multiple and the initial multiple single-step variation, and take the second actual adjustment multiple as a current actual adjustment multiple;

Step S1033: Adjust the signal value at the first sampling point based on the current actual adjustment multiple; and

Step S1034: Determine a next multiple single-step variation based on the target adjustment multiple and the current actual adjustment multiple, determine a next actual adjustment multiple based on the current actual adjustment multiple and the next multiple single-step variation, adjust a signal value at a next sampling point based on the next actual adjustment multiple, then take the next actual adjustment multiple as a current actual adjustment multiple, and repeat the step of determining a next multiple single-step variation based on the target adjustment multiple and the current actual adjustment multiple until at least one first preset condition is satisfied;

wherein the first preset condition includes: the current actual adjustment multiple belongs to a first target interval, wherein the first target interval is determined by the target adjustment multiple; and a window end time to reach the current peak-to-peak value adjustment time window.

In this example, the first target interval is an allowable error range of the target adjustment multiple, which will not be described in detail herein.

If the current actual adjustment multiple belongs to the first target interval and the window end time of the current peak-to-peak value adjustment time window is not reached, signal values of the original human biological signal at the remaining sampling points within the current peak-to-peak value adjustment time window are adjusted based on the current actual adjustment multiple until the window end time of the current peak-to-peak value adjustment time window is reached.

Based on the above-mentioned method, except that the initial actual adjustment multiple is manually set, each actual adjustment multiple can be calculated according to the two parameters of the previous actual adjustment multiple and the single-step variation of the previous multiple jointly determined by the previous actual adjustment multiple and the target adjustment multiple, and the previous actual adjustment multiple refers to the actual adjustment multiple corresponding to the previous sampling point.

It should be noted that the initial actual adjustment multiple does not correspond to any sampling point, but is merely an initial value of the preset actual adjustment multiple, and the second actual adjustment multiple obtained based on the initial multiple single-step variation and the initial actual adjustment multiple corresponds to the first sampling point within the current peak-to-peak value adjustment time window.

The initial actual adjustment multiple is generally set as 1, and the actual adjustment multiple needs to increase from 1, so that it becomes closer to the target adjustment multiple; therefore, the calculation formula for the single-step variation of factor and the actual adjustment multiple can be respectively set as:

aᵢ=(c−bᵢ)/(tᵢ*f) Equation (1)

bᵢ₊₁=bᵢ+aᵢ Equation (2)

where aᵢ represents the multiple single-step variation of the i^{th} sampling point; c represents the target adjustment multiple; bᵢ represents the actual adjustment multiple of the i^{th} sampling point; bᵢ₊₁ represents the actual adjustment multiple of the (i+1)^{th} sampling point; t₁ represents time in seconds; f represents the signal sampling rate in Hertz.

According to the above-mentioned formulas (1) to (2), the actual adjustment multiple and the value of single-step variation of multiple corresponding to each sampling point in the current peak-to-peak value adjustment time window can be obtained, see Table 1 for details.

TABLE 1

| | Parameter | | |
|---|---|---|---|
| | Target | Actual | Multiple |
| Sampling | adjustment | adjustment | single-step |
| point | multiple c | multiple bᵢ | variation aᵢ |
| 0 | c | b₀ = 1 | a₀ = (c − b₀)/(t₁*f) |
| 1 | c | b1 = b₀ + a₀ | a₁ = (c − b₁)/(t₁*f) |
| 2 | c | b₂ = b₁ + a₁ | a₂ = (c − b₂)/(t₁*f) |
| . . . . . . | . . . . . . | . . . . . . | . . . . . . |
| i | c | bᵢ = bᵢ₋₁ + aᵢ₋₁ | aᵢ = (c − bᵢ)/(t₁*f) |
| i + 1 | c | bᵢ₊₁ = bᵢ + aᵢ | aᵢ₊₁ = (c − bᵢ₊₁)/(t₁*f) |

It can be seen from Table 1 that as the value of the actual adjustment multiple increases, the value of the multiple single-step variation decreases continuously, and when the value of the multiple single-step variation decreases to or approaches 0, the value of the actual adjustment multiple increases to or approaches the target adjustment multiple.

Furthermore, as the single-step variation of the multiples becomes smaller and smaller, the change of the actual adjustment multiples becomes smaller and smaller, so the change trend of the waveform becomes more and more gentle.

When the target adjustment multiple c is greater than 1, a₀ is a positive number, so that b₁>b₀, at this moment, the original signal value at the first sampling point is amplified, and the signal value becomes b1 times of the original value; when bᵢ increases to be greater than c and does not belong to the first target interval, bᵢ is a negative number, so that bᵢ, then the original signal value of the (i+1)^{th} sampling point is amplified, and the signal value becomes bᵢ₊₁ times of the original value.

It can be seen that when the actual adjustment multiple exceeds the target adjustment multiple too much, the value of the actual adjustment multiple is adjusted downward by changing the multiple single-step variation to a negative number, and is adjusted downward to the first target interval one or more times, so that the actual adjustment multiple reaches the optimal value and does not change any more.

When the target adjustment multiple c is greater than 0 and less than 1, a₀ is a negative number, so that 0<b₁<b₀, at this moment, the original signal value at the first sampling point is reduced, and the signal value becomes bᵢ times of the original value; when bᵢ is reduced to be smaller than c and does not belong to the first target interval, aᵢ is a positive number, so that bᵢ₊₁>bᵢ, then the original signal value of the (i+1)^{th} sampling point is reduced, and the signal value becomes twice the original bᵢ₊₁.

It can be seen that when the actual adjustment multiple is less than the target adjustment multiple too much, the value of the actual adjustment multiple is adjusted up by changing the multiple single-step variation to a positive number, and is adjusted up to the first target interval one or more times, so that the actual adjustment multiple reaches the optimum and does not change any more.

It should be noted that t₁ in the above equations (1) to (2) is generally the same as or different from the duration setting of the peak-to-peak value adjustment time window. t₁ represents how long it takes to adjust the original peak-peak value of the original human biological signal to the target peak-peak value; therefore, the larger t₁ is, the more slowly the original human biological signal changes, and the smaller t₁ is, the more quickly the original human biological signal changes. Therefore, in order to better suppress waveform distortion, t₁ is generally set to not less than 2 seconds.

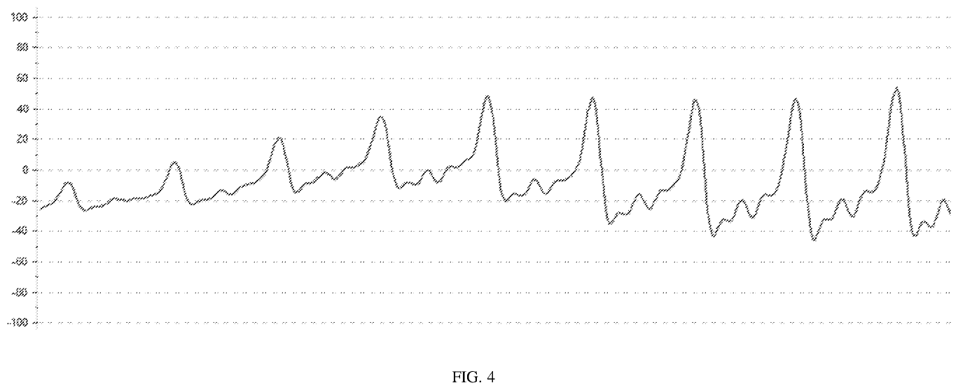

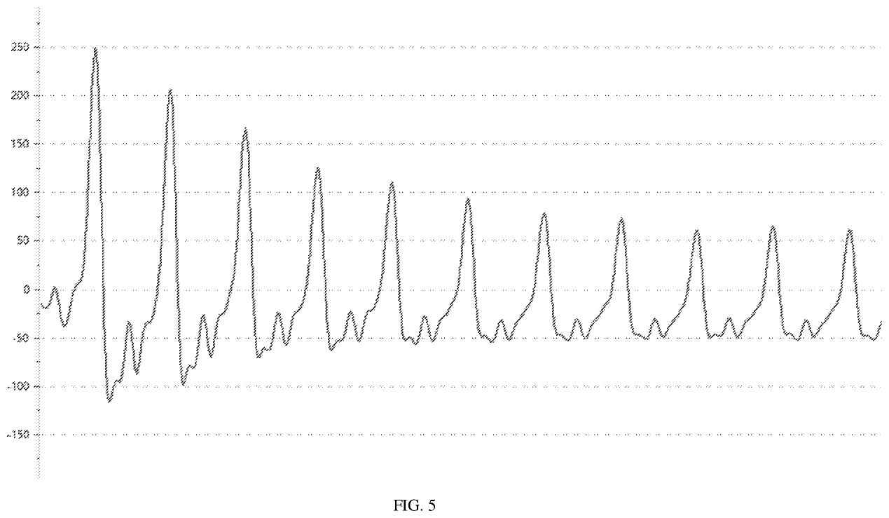

The peak-to-peak value of the weak or excessively strong original human biological signal can be gradually adjusted to a suitable degree with the display area of the waveform display device by the above-mentioned waveform processing method, and FIGS. 4 and 5 successively show schematic diagrams for processing the weak or excessively strong original human biological signal by the above-mentioned waveform processing method.

For ECG signal, the thorax changes caused by human respiration, leads to offset of ECG electrodes, causing a baseline drift. However, because the baseline drift is close to the ST segment of ECG signal, if it is not properly processed, it will cause the ST segment of ECG signal distortion, resulting in misdiagnosis.

For the PPG signal, when the PPG signal is measured by Functional Near-infrared Spectroscopy (FNIRS), the detection circuit contains a DC offset, while the PPG signal itself is an AC variable. The superimposed DC offset will make the baseline of the PPG signal very high, thus making the PPG waveform in an unobvious state. In addition, when the human body performs some relatively slow activities or the ambient temperature slowly decreases, the change of human blood volume will also cause the baseline of PPG signal to change, so that the PPG waveform will present an insignificant state.

It can be seen that the original human biological signal may have baseline drift, so that the whole waveform cannot be displayed well. In order to solve the problem of baseline wandering, the signal baseline is usually adjusted to the target baseline position directly, but this will make the waveform position abrupt, resulting in waveform distortion and misdiagnosis.

In order to solve the problem of waveform distortion caused by an instantaneous offset of a waveform baseline, the embodiment of the present application is to realize a step-by-step offset of a baseline of an original human biological signal; therefore, a plurality of baseline adjustment time windows are used to perform traversal sampling on the original human biological signal, i.e., the original human biological signal is segmented according to time, and the baseline of the original physiological signal within each baseline adjustment time window is adjusted in sequence.

For two adjacent baseline adjustment time windows, the end time of the previous baseline adjustment time window is the start time of the next baseline adjustment time window. The duration of the baseline adjustment time window and the peak-to-peak value adjustment time window may be the same or different; also, the duration of each baseline adjustment time window may be the same or different, as the same for the peak-to-peak value adjustment time window.

Similar to the signal value processing method described above, the baseline offset method also discards the signal processing for the first baseline adjustment time window, which simply provides the original baseline value for the second baseline adjustment time window. However, the baseline offset processing method for the original human biological signal of any baseline adjustment time window except the first baseline adjustment time window is the same.

The waveform processing method for the original human biological signal of any baseline adjustment time window other than the first baseline adjustment time window is specifically described below, wherein any baseline adjustment time window is used as the current baseline adjustment time window.

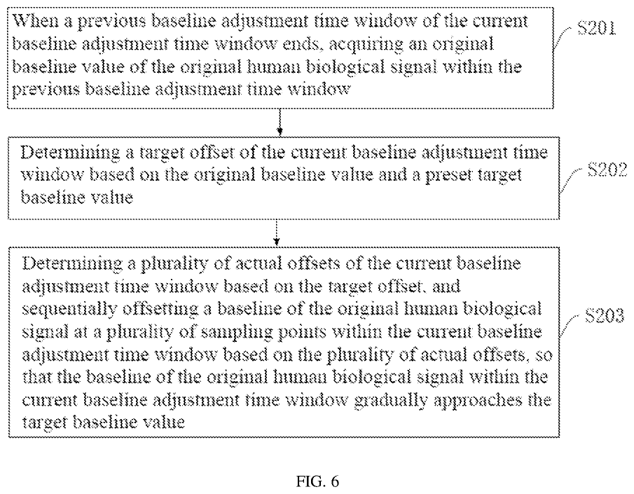

FIG. 6 is a flowchart showing another method for processing human biological signal data. As shown in FIG. 6, the main flow of the method is described as follows (steps S201 to S203):

Step S201: When a previous baseline adjustment time window of the current baseline adjustment time window ends, acquire an original baseline value of the original human biological signal within the previous baseline adjustment time window;

In Step S201, at the end of each baseline adjustment time window, the minimum value of the original human biological signal within the baseline adjustment time window is calculated and stored in a buffer with a fixed length; in order to stabilize the data and filter out outliers, sliding median filtering and sliding mean filtering are performed on the minimum value stored in the buffer to obtain the original baseline value of the original human biological signal within the baseline adjustment time window, and the window mark is cleared to prepare for starting the next baseline adjustment time window.

Since the original baseline value is calculated as the minimum value of the original human biological signal within the previous baseline adjustment time window, it is necessary to ensure that the baseline adjustment time window can find a complete signal period. Based on the above-mentioned content regarding the value of the duration of the peak-to-peak value adjustment time window, the duration of the baseline adjustment time window can also be set to not less than 2 seconds to ensure that there must be a complete signal period within the baseline adjustment time window, and the minimum value of the original human biological signal can be obtained.

Step S202: Determine a target offset of the current baseline adjustment time window based on the original baseline value and a preset target baseline value;
in the Step S202, the target baseline value is manually set, and a default value is generally 0; however, other values may be set, to which this embodiment is not particularly limited.

The target offset is the difference between the target baseline value and the original baseline value. If the original baseline value is less than the target baseline value, i.e., the target offset is greater than 0, then the baseline position within the current baseline adjustment time window needs to be moved upwards; if the original baseline value is greater than the target baseline value, i.e., the target offset is less than 0, then the baseline position within the current baseline adjustment time window needs to be moved downwards; if the original baseline value is equal to the target baseline value, i.e. the target offset is equal to zero, then there is no need to offset the baseline position within the current baseline adjustment time window.

Step S203: Determine a plurality of actual offsets of the current baseline adjustment time window based on the target offset, and sequentially offset a baseline of the original human biological signal at a plurality of sampling points within the current baseline adjustment time window based on the plurality of actual offsets, so that the baseline of the original human biological signal within the current baseline adjustment time window gradually approaches the target baseline value.

In order to make the baseline value of the original human biological signal in the current baseline adjustment time window gradually approach the target baseline value, a plurality of actual offsets can be determined according to the number of sampling points in the current baseline adjustment time window, so that the plurality of actual offsets correspond to the signal values at the plurality of sampling points one by one, that is to say, when the sampling time corresponding to each sampling point in the current baseline adjustment time window is reached, the signal baseline of the sampling point is offset according to the actual offset corresponding to the sampling point.

For the previous sampling point in the current baseline adjustment time window, the absolute value of the corresponding actual offset shall not be greater than the absolute value of the target offset, and the overall change trend of the actual offset shall gradually approach the target offset, for example: as a function of time, the absolute values of the plurality of actual offsets may exhibit a gradually increasing trend. Of course, it may also occur that the absolute value of the actual offset corresponding to certain sampling points fluctuates upwards and downwards.

No matter whether the actual offset corresponding to the previous sampling point in the current baseline adjustment time window reaches the target offset, as long as the current baseline adjustment time window ends, it is necessary to acquire an original baseline value according to the original human biological signal in the current baseline adjustment time window, determine the target offset of the next time window according to the original baseline value and the target baseline value, and continue offsetting the baseline in the next time window according to the new target offset. That is to say, for different sampling points of the same baseline adjustment time window, the target offset thereof is constant; however, for the sampling points of different baseline adjustment time windows, the target offset is generally changed, because the original baseline value may be different, but if the waveform of the original human biological signal is relatively stable, the target offset of each baseline adjustment time window may also be almost unchanged.

If the actual offset reaches the target offset when the sampling moment of the previous sampling point in the current baseline adjustment time window is not reached, then the actual offsets corresponding to the subsequent sampling points are all set as the target offset until the end of the current baseline adjustment time window.

It should be noted that the sampling time of the previous sampling point may be the end time of the current baseline adjustment time window or a time before the end of the current baseline adjustment time window.

It should be noted that, in consideration of the error factor, when the actual offset amount belongs to the error range of the target offset amount, it can be determined that the actual offset amount reaches the target offset amount. For example: if the target offset is 10 and the error range thereof is ±0.1, then when the actual offset belongs to [9.9, 10.1], it is determined that the actual offset reaches the target offset.

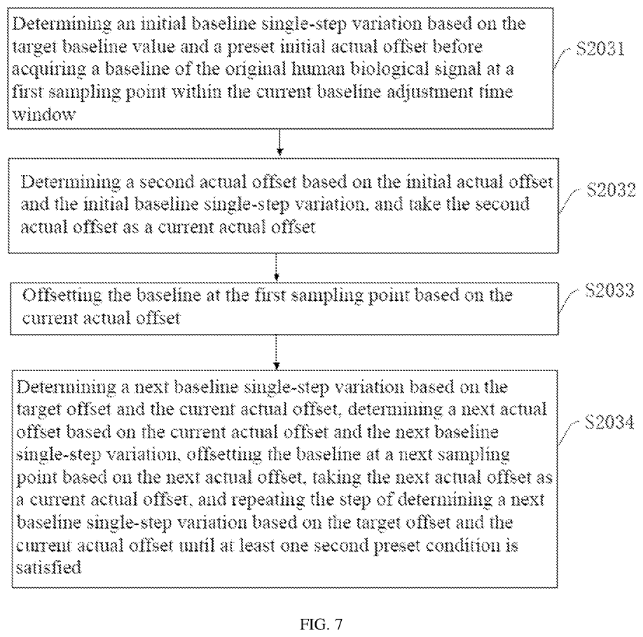

As an example to this embodiment, each actual offset may be set by setting the baseline single-step variation, so that the actual offset gradually approaches the target offset, thereby achieving that the baseline of the original human biological signal within the current peak-to-peak value adjustment time window gradually approaches the target baseline value. As shown in FIG. 7, the specific steps of Step S203 are as follows:

Step S2031: Determine an initial baseline single-step variation based on the target baseline value and a preset initial actual offset before acquiring a baseline at a first sampling point of the original human biological signal within the current baseline adjustment time window;

Step S2032: Determine a second actual offset based on the initial actual offset and the initial baseline single-step variation, and take the second actual offset as a current actual offset;

Step S2033: Offset the baseline at the first sampling point based on the current actual offset;

Step S2034: Determine a next baseline single-step variation based on the target offset and the current actual offset, determine a next actual offset based on the current actual offset and the next baseline single-step variation, offset a baseline at a next sampling point based on the next actual offset, then take the next actual offset as a current actual offset, and repeat the step of determining a next baseline single-step variation based on the target offset and the current actual offset until at least one second preset condition is satisfied;

wherein the second preset condition includes: the current actual offset belongs to a second target interval, wherein the second target interval is determined by the target offset; and reach a window end time of the current baseline adjustment time window.

In this example, the second target interval is an allowable error range of the target offset, and will not be described in detail herein.

If the current actual offset belongs to the second target interval and the window end time of the current baseline adjustment time window is not reached, the baseline of the original human biological signal at the remaining sampling points within the current baseline adjustment time window is offset based on the current actual offset until the window end time of the current baseline adjustment time window is reached.

Based on the above-mentioned method, except that the initial actual offset is manually set, each actual offset can be calculated according to the two parameters of the previous actual offset and the previous baseline single-step variation quantity jointly determined by the previous actual offset and the target offset, and the previous actual offset refers to the actual offset corresponding to the previous sampling point.

It should be noted that the initial actual offset does not correspond to any sampling point, but is merely an initial value of the preset actual offset, and the second actual offset obtained based on the initial baseline single-step variation and the initial actual offset corresponds to the first sampling point within the current baseline adjustment time window.

The initial actual offset is generally set as 0, and the absolute value of the actual offset needs to increase from 0, so that it is closer and closer to the target offset; therefore, the calculation formula for the single-step variation of the baseline and the actual offset can be respectively set as:

dⱼ=(g−eⱼ)/(t₂*f) equation (3)

eⱼ₊₁=eⱼ+dⱼ equation (4)
wherein dᵢ represents the baseline single-step variation at the i^{th} sampling point; g represents a target offset; eⱼ represents the actual offset of the i^{th} sampling point; eⱼ₊₁ represents the actual offset of the (j+1)^{th} sampling point; t₂ represents time in seconds; and f represents the signal sampling rate in Hertz.

According to the above-mentioned equations (3) to (4), the actual offset corresponding to each sampling point in the current baseline adjustment time window and the value of the baseline single-step variation can be obtained, see Table 2 for details.

TABLE 2

| | Parameter | | |
|---|---|---|---|
| Sampling | | | Baseline single-step |
| point | Target offset g | Actual offset eⱼ | variation dⱼ |
| 0 | g | e₀ = 0 | d₀ = (g − e₀)/(t₂*f) |
| 1 | g | e₁ = e₀ + d₀ | d₁ = (g − e₁)/(t₂*f) |
| 2 | g | e₂ = e₁ + d₁ | d₂ = (g − e₂)/(t₂*f) |
| . . . . . . | . . . . . . | . . . . . . | . . . . . . |
| j | g | eⱼ = eⱼ₋₁ + dⱼ₋₁ | dⱼ = (g − eⱼ)/(t₂*f) |
| j + 1 | g | eⱼ₊₁ = eⱼ + dⱼ | dⱼ₊₁ = (g − eⱼ₊₁)/(t₂*f) |

It can be seen from Table 2 that as the absolute value of the actual offset increases, the absolute value of the baseline single-step variation decreases, and when the value of the baseline single-step variation decreases to or approaches 0, the value of the actual offset increases to or approaches the target offset.

Furthermore, as the variation of the baseline in a single-step becomes smaller, the change of the actual offset becomes smaller, so the change trend of the waveform baseline becomes more and more gentle.

When the target offset g is greater than 0, d₀ is a positive number, so that e₁>e₀>0, and at this moment, the original baseline position at the first sampling point is moved upwards by e₁; when eⱼ increases to be greater than g and does not belong to the second target interval, dⱼ is a negative number, such that 0<eⱼ₊₁<eⱼ, then the original baseline position of the (j+1)^{th} sampling point is offset upwards by eⱼ₊₁, and the signal baseline position of the (j+1)^{th} sampling point is lower than the signal baseline position of the (j)^{th} sampling point.

It can be seen that when the actual offset exceeds the target offset by too much, the value of the actual offset is adjusted downward by changing the baseline single-step variation to a negative number, and is adjusted downward to the second target interval one or more times, so that the actual offset reaches the optimum and does not change any more.

When the target offset is less than 0, d₀ is a negative number, so that e₁<e₀<0, and at this moment, the original baseline position at the first sampling point is moved down by e₁; when eⱼ decreases to be smaller than g and does not belong to the second target interval, dⱼ is a positive number, such that eⱼ<eⱼ+₁<0, then the original baseline position of the (j+1)^{th} sampling point is offset downwards by eⱼ₊₁, and the signal baseline position of the (j+1)^{th} sampling point is higher than the signal baseline position of the (j)^{th} sampling point.

It can be seen that when the actual offset is too much smaller than the target offset, the value of the actual offset is adjusted up by changing the baseline single-step variation to a positive number, and is adjusted up to the second target interval one or more times, so that the actual offset reaches the optimum and does not change any more.

It should be noted that t₂ in the above equations (3) to (4) is generally the same as or different from the duration setting of the baseline adjustment time window. t₂ represents how long it takes to adjust the original baseline value of the original human biological signal to the target baseline value; therefore, the larger t₂ is, the slower the original human biological signal changes, and the smaller t₂ is, the faster the original human biological signal changes. Therefore, in order to better suppress waveform distortion, t₂ is generally set to not less than 2 seconds.

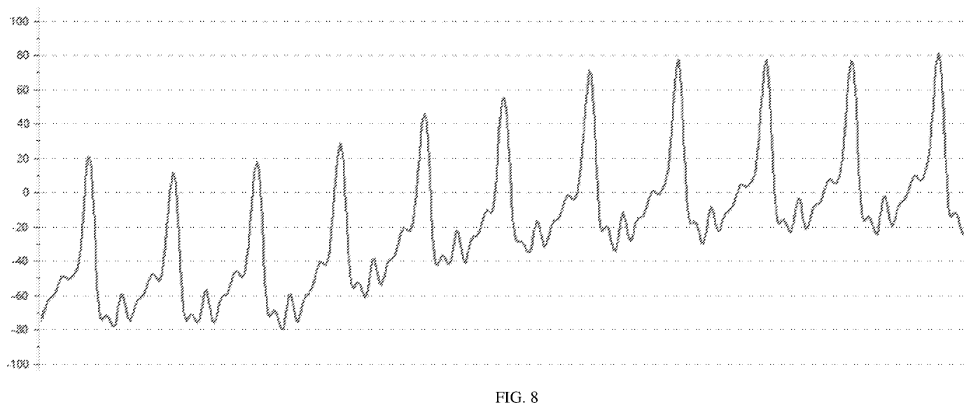

The baseline of the original human biological signal can be gradually adjusted to a suitable position in the display area of the waveform display device by the above waveform processing method, and FIG. 8 shows a schematic diagram of the adjustment of the baseline of the original human biological signal by the above waveform processing method.

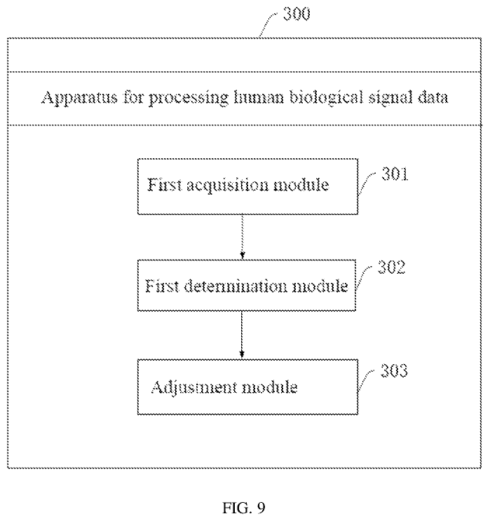

FIG. 9 is a block diagram of an apparatus for processing human biological signal data 300 according to an embodiment of the present application. For the original human biological signal in any one of the plurality of peak-to-peak value adjustment time windows except a first peak-to-peak value adjustment time window, the any one of the peak-to-peak value adjustment time windows is taken as a current peak-to-peak value adjustment time window. As shown in FIG. 9, the apparatus for processing human biological signal data 300 mainly includes:

a first acquisition module 301 configured to, when a previous peak-to-peak value adjustment time window of the current peak-to-peak value adjustment time window ends, acquire an original peak-to-peak value of the original human biological signal within the previous peak-to-peak value adjustment time window;

a first determination module 302 configured to determine a target adjustment multiple of a current peak-to-peak value adjustment time window based on the original peak-to-peak value and a preset target peak-to-peak value; and

an adjustment module 303 configured to determine a plurality of actual adjustment multiples of the current peak-to-peak value adjustment time window based on the target adjustment multiple, and sequentially adjust signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the target peak-to-peak value; wherein the plurality of actual adjustment multiples correspond to the signal values at the plurality of sampling points one by one.

As an example to this embodiment, the adjustment module 303 is specifically configured to determine a plurality of actual adjustment multiples of the current peak-to-peak value adjustment time window based on the target adjustment multiple, and sequentially adjust signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the target peak-to-peak value; wherein the plurality of actual adjustment multiples correspond to the signal values at the plurality of sampling points one by one; the adjustment module is further configured to determine an initial multiple single-step variation based on the target adjustment multiple and a preset initial actual adjustment multiple before acquiring a signal value of the original human biological signal at a first sampling point within the current peak-to-peak value adjustment time window; determine a second actual adjustment multiple based on the initial actual adjustment multiple and the initial multiple single-step variation, and take the second actual adjustment multiple as a current actual adjustment multiple; adjust the signal value at the first sampling point based on the current actual adjustment multiple; determine a next multiple single-step variation based on the target adjustment multiple and the current actual adjustment multiple, determine a next actual adjustment multiple based on the current actual adjustment multiple and the next multiple single-step variation, adjust a signal value at a next sampling point based on the next actual adjustment multiple, then take the next actual adjustment multiple as the current actual adjustment multiple, and repeat the step of determining a next multiple single-step variation based on the target adjustment multiple and the current actual adjustment multiple until at least one first preset condition is satisfied; wherein the first preset condition comprises: the current actual adjustment multiple belongs to a first target interval, wherein the first target interval is determined by the target adjustment multiple; and a window end time of the current peak-to-peak value adjustment time window is reached.

In this example, the adjustment module 303 is further specifically configured to, if the current actual adjustment multiple belongs to the first target interval and the window end time of the current peak-to-peak value adjustment time window is not reached, signal values of the original human biological signal at the remaining sampling points within the current peak-to-peak value adjustment time window are adjusted based on the current actual adjustment multiple until the window end time of the current peak-to-peak value adjustment time window is reached.

As an example to this embodiment, the adjustment module 303 sets the plurality of actual adjustment times to be in an increasing trend over time.

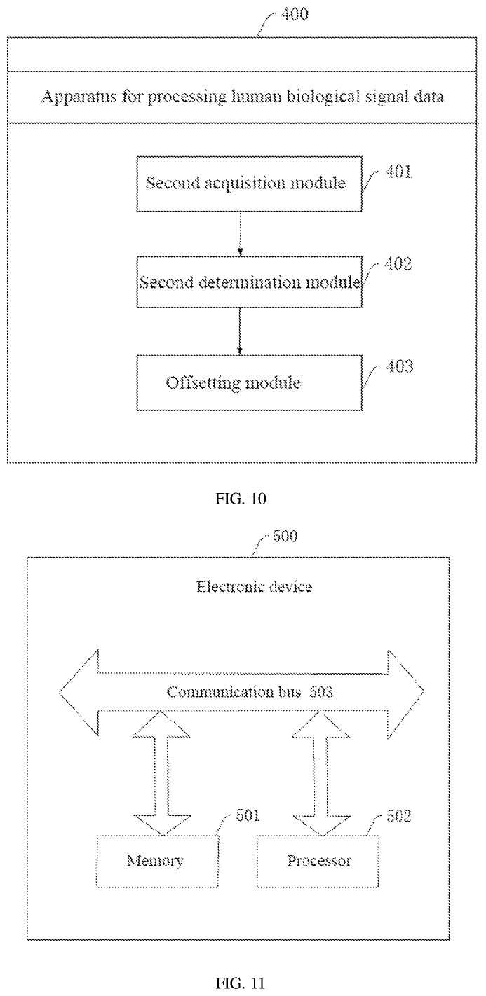

FIG. 10 is a block diagram of an apparatus for processing human biological signal data 400 according to an embodiment of the present application. An original human biological signal is traversed with a plurality of preset baseline adjustment time windows; and for the original human biological signal in any one of the plurality of baseline adjustment time windows except a first baseline adjustment time window, the any one of the baseline adjustment time windows is taken as a current baseline adjustment time window. As shown in FIG. 10, the apparatus for processing human biological signal data 400 mainly includes:

a second acquisition module 401 configured to, when a previous baseline adjustment time window of the current baseline adjustment time window ends, acquire an original baseline value of the original human biological signal within the previous baseline adjustment time window;

a second determination module 402 configured to determine a target offset of the current baseline adjustment time window based on the original baseline value and a preset target baseline value; and

an offsetting module 403 configured to determine a plurality of actual offsets of the current baseline adjustment time window based on the target offset, and sequentially offset a baseline of the original human biological signal at a plurality of sampling points within the current baseline adjustment time window based on the plurality of actual offsets, so that the baseline of the original human biological signal within the current baseline adjustment time window gradually approaches the target baseline value.

As an example to this embodiment, an offsetting module 403 is specifically configured to determine a plurality of actual offsets of the current baseline adjustment time window based on the target offset, and sequentially offset a baseline of the original human biological signal at a plurality of sampling points within the current baseline adjustment time window based on the plurality of actual offsets, so that the baseline of the original human biological signal within the current baseline adjustment time window gradually approaches the target baseline value; wherein the plurality of actual offsets correspond to a baseline at the plurality of sampling points one by one; determine an initial baseline single-step variation based on the target baseline value and a preset initial actual offset before acquiring the baseline of the original human biological signal at a first sampling point within the current baseline adjustment time window; determine a second actual offset based on the initial actual offset and the initial baseline single-step variation, and take the second actual offset as a current actual offset; offset the baseline at the first sampling point based on the current actual offset; determine a next baseline single-step variation based on the target offset and the current actual offset, determine a next actual offset based on the current actual offset and the next baseline single-step variation, offset the baseline at the next sampling point based on the next actual offset, then take the next actual offset as the current actual offset, and repeat the step of determining a next baseline single-step variation based on the target offset and the current actual offset until at least one second preset condition is satisfied; wherein the second preset condition comprises: the current actual offset belongs to a second target interval, wherein the second target interval is determined by the target offset; and a window end time of the current baseline adjustment time window is reached.

As an example to this embodiment, the offsetting module 403 is further specifically configured to, if the current actual offset belongs to the second target interval and the window end time of the current baseline adjustment time window is not reached, offset the baseline of the original human biological signal at the remaining sampling points within the current baseline adjustment time window based on the current actual offset until the window end time of the current baseline adjustment time window is reached.

Each functional module in the embodiments of the present application can be integrated together to form an independent unit, such as being integrated into a processing unit, or each module can be physically present separately, or two or more modules can be integrated to form an independent unit. The above-mentioned integrated units may be implemented in the form of hardware or in the form of software functional units. If implemented in the form of a software function module and sold or used as a stand-alone product, the function may be stored in a computer-readable storage medium. Based on such an understanding, the technical solution of the present application, either in itself or in part contributing to the prior art, may be embodied in the form of a software product stored in a storage medium including instructions to cause an electronic device (which may be a personal computer, server or network device, etc.) or processor to perform all or part of the steps of the methods of the various embodiments of the present application. The storage medium includes: various media may store the program code, such as a U-disk, removable hard disk, read-only memory, random access memory, magnetic or optical disk.

Various modifications and specific examples of the method provided in the embodiments of the present application are also applicable to an apparatus for processing human biological signal data provided in the embodiments. From the above-mentioned detailed description of the method for processing human biological signal data, a person skilled in the art can clearly know the implementation method for the apparatus for processing human biological signal data in the embodiments, which will not be described in detail herein for the sake of brevity.

FIG. 11 is a block diagram of an electronic device 500 according to an embodiment of the present application. As shown in FIG. 11, an electronic device 500 includes a memory 501, a processor 502 and a communication bus 503; the memory 501 and the processor 502 are connected via a communication bus 503.

The memory 501 may be used to store instructions, programs, code, sets of code, or sets of instructions. The memory 501 may include a stored program area and a stored data area, wherein the stored program area may store instructions for implementing an operating system, instructions for at least one function, instructions for implementing the method for processing human biological signal data provided in the above-mentioned embodiment, etc.; the storage data area may store data or the like involved in the method for processing human biological signal data provided in the above embodiment.

The processor 502 may include one or more processing cores. The processor 502 performs various functions and processes data of the present application by running or executing instructions, programs, code sets, or instruction sets stored in memory 501 to invoke data stored in the memory 501. The processor 502 may be at least one of an Application Specific Integrated Circuit (ASIC), a Digital Signal Processor (DSP), a Digital Signal Processing Device (DSPD), a Programmable Logic Device (PLD), a Field Programmable Gate Array (FPGA), a Central Processing Unit (CPU), a controller, a microcontroller, and a microprocessor. It will be appreciated that the electronics used to implement the functions of the processor 502 described above may be other for different devices and that the embodiments of the present application are not particularly limited.

The communication bus 503 may include a path to transfer information between the described above. The communication bus 503 may be a Peripheral Component Interconnect (PCI) bus or an Extended Industry Standard Architecture (PCI) bus or the like. The communication bus 503 may be divided into an address bus, a data bus, a control bus, etc. For ease of illustration, only one double-headed arrow is shown in FIG. 11, but does not indicate that there is only one bus or type of bus. In addition, the electronic device shown in FIG. 11 is only one example and should not impose any limitation on the functionality and scope of use of the embodiments of the present application.

Embodiments of the present application provide a computer-readable storage medium storing a computer program to be loaded by a processor and executing the method for processing human biological signal data as provided in the above embodiments.

In this embodiment, a computer-readable storage medium may be a tangible device that retains and stores instructions for use by an instruction execution device. The computer-readable storage medium may be but not limited to, an electrical storage device, a magnetic storage device, an optical storage device, an electromagnetic storage device, a semiconductor storage device, or any combination of the above. In particular, the computer-readable storage medium may be a portable computer diskette, a hard disk, a U-disk, a random access memory (RAM), a read-only memory (ROM), an erasable programmable read-only memory (EPROM or Flash memory), a SRAM, a portable compact disc read-only memory (CD-ROM), a digital versatile disc (DVD), a memory stick, a floppy disk, an optical disk, a magnetic disk, a mechanical encoding device, and any combination thereof.

The computer program in this embodiment includes program code for performing the method shown in FIG. 2, FIG. 3, FIG. 6, FIG. 7, and the program code may include instructions corresponding to performing the method steps provided by the above embodiments. The computer program may be downloaded from a computer-readable storage medium to various computing/processing devices, or from an external computer or external storage device over a network, such as the Internet, a local area network, a wide area network, and/or a wireless network. The computer program may execute entirely on the user's computer as a stand-alone software package.

In the examples provided herein, it should be understood that the disclosed systems, devices, and methods may be implemented in other ways. For example, the apparatus embodiments described above are merely illustrative, e.g. partitioning of modules or units, partitioning of only one logical function, actual implementation may have additional partitioning, e.g. multiple units or components may be combined or integrated into another system, or some features may be omitted, or not performed. In another aspect, the couplings or direct couplings or communication connections shown or discussed with respect to each other may be indirect couplings or communication connections through some interface, apparatus, or unit, and may be electrical, mechanical, or otherwise.

In addition, it is to be understood that relational terms such as first and second, and the like, are used solely to distinguish one entity or action from another entity or action without necessarily requiring or implying any actual such relationship or order between such entities or actions. Further, the terms “include”, “comprise”, or any other variation thereof, are intended to cover a non-exclusive inclusion, such that a process, method, article, or apparatus that includes a list of elements does not include only those elements but may include other elements not expressly listed or inherent to such process, method, article, or apparatus.

The above description is only a preferred embodiment of the present application and is not intended to limit the present application, and various modifications and changes may be made to the present application by a person skilled in the art. Any modification, equivalent replacement, or improvement made within the spirit and principle of the present application shall be included in the protection scope of the present application.

What is claimed is:

1. A method for processing human biological signal data, wherein an original human biological signal is traversed with a plurality of preset peak-to-peak value adjustment time windows; for the original human biological signal in any one of the plurality of preset peak-to-peak value adjustment time windows except a first preset peak-to-peak value adjustment time window, the any one of the plurality of preset peak-to-peak value adjustment time windows is taken as a current peak-to-peak value adjustment time window, and the method comprises:
when a previous peak-to-peak value adjustment time window of the current peak-to-peak value adjustment time window ends, acquiring an original peak-to-peak value of the original human biological signal within the previous peak-to-peak value adjustment time window;
determining a target adjustment multiple of the current peak-to-peak value adjustment time window based on the original peak-to-peak value and a preset target peak-to-peak value; and
determining a plurality of actual adjustment multiples of the current peak-to-peak value adjustment time window based on the target adjustment multiple, and sequentially adjusting signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the preset target peak-to-peak value;
wherein the plurality of actual adjustment multiples correspond to the signal values at the plurality of sampling points one by one.

2. The method according to claim 1, wherein the determining a plurality of actual adjustment multiples of the current peak-to-peak value adjustment time window based on the target adjustment multiple, and sequentially adjusting signal values of the original human biological signal at a plurality of sampling points within the current peak-to-peak value adjustment time window based on the plurality of actual adjustment multiples, comprises:
determining an initial multiple single-step variation based on the target adjustment multiple and a preset initial actual adjustment multiple before acquiring a signal value of the original human biological signal at a first sampling point within the current peak-to-peak value adjustment time window;
determining a second actual adjustment multiple based on the preset initial actual adjustment multiple and the initial multiple single-step variation, and taking the second actual adjustment multiple as a current actual adjustment multiple;
adjusting the signal value at the first sampling point based on the current actual adjustment multiple; and
determining a next multiple single-step variation based on the target adjustment multiple and the current actual adjustment multiple, determining a next actual adjustment multiple based on the current actual adjustment multiple and the next multiple single-step variation, adjusting a signal value at a next sampling point based on the next actual adjustment multiple, then taking the next actual adjustment multiple as the current actual adjustment multiple, and repeating the step of determining a next multiple single-step variation based on the target adjustment multiple and the current actual adjustment multiple until at least one first preset condition is satisfied;
wherein the at least one first preset condition comprises:
the current actual adjustment multiple belongs to a first target interval, wherein the first target interval is determined by the target adjustment multiple; and
a window end time of the current peak-to-peak value adjustment time window is reached.

3. The method according to claim 2, wherein when the current actual adjustment multiple belongs to the first target interval and the window end time of the current peak-to-peak value adjustment time window is not reached, signal values of the original human biological signal at remaining sampling points within the current peak-to-peak value adjustment time window are adjusted based on the current actual adjustment multiple until the window end time of the current peak-to-peak value adjustment time window is reached.

4. The method according to claim 1, wherein the plurality of actual adjustment multiples exhibit an increasing trend over time.

5. The method according to claim 1, wherein an original human biological signal is traversed with a plurality of preset baseline adjustment time windows; and for the original human biological signal in any one of the plurality of preset baseline adjustment time windows except a first preset baseline adjustment time window, the any one of the plurality of preset baseline adjustment time windows is taken as a current baseline adjustment time window, and the method further comprises:
when a previous baseline adjustment time window of the current baseline adjustment time window ends, acquiring an original baseline value of the original human biological signal within the previous baseline adjustment time window;
determining a target offset of the current baseline adjustment time window based on the original baseline value and a preset target baseline value; and
determining a plurality of actual offsets of the current baseline adjustment time window based on the target offset, and sequentially offsetting a baseline of the original human biological signal at a plurality of sampling points within the current baseline adjustment time window based on the plurality of actual offsets, so that the baseline of the original human biological signal within the current baseline adjustment time window gradually approaches the preset target baseline value;
wherein the plurality of actual offsets correspond to the baseline at the plurality of sampling points one by one.

6. The method according to claim 5, wherein the determining a plurality of actual offsets of the current baseline adjustment time window based on the target offset, and sequentially offsetting a baseline of the original human biological signal at a plurality of sampling points within the current baseline adjustment time window based on the plurality of actual offsets, comprises:
determining an initial baseline single-step variation based on the preset target baseline value and a preset initial actual offset before acquiring a baseline of the original human biological signal at a first sampling point within the current baseline adjustment time window;
determining a second actual offset based on the preset initial actual offset and the initial baseline single-step variation, and taking the second actual offset as a current actual offset;
offsetting the baseline at the first sampling point based on the current actual offset; and
determining a next baseline single-step variation based on the target offset and the current actual offset, determining a next actual offset based on the current actual offset and the next baseline single-step variation, offsetting the baseline at a next sampling point based on the next actual offset, then taking the next actual offset as the current actual offset, and repeating the step of determining a next baseline single-step variation based on the target offset and the current actual offset until at least one second preset condition is satisfied;
wherein the at least one second preset condition comprises:
the current actual offset belongs to a second target interval, wherein the second target interval is determined by the target offset; and
a window end time of the current baseline adjustment time window is reached.

7. The method according to claim 6, wherein when the current actual offset belongs to the second target interval and the window end time of the current baseline adjustment time window is not reached, the baseline of the original human biological signal at remaining sampling points within the current baseline adjustment time window is offset based on the current actual offset until the window end time of the current baseline adjustment time window is reached.

8. A method for processing human biological signal data, wherein an original human biological signal is traversed with a plurality of preset baseline adjustment time windows; and for the original human biological signal in any one of the plurality of preset baseline adjustment time windows except a first present baseline adjustment time window, the any one of the plurality of preset baseline adjustment time windows is taken as a current baseline adjustment time window, and the method comprises:
when a previous baseline adjustment time window of the current baseline adjustment time window ends, acquiring an original baseline value of the original human biological signal within the previous baseline adjustment time window;
determining a target offset of the current baseline adjustment time window based on the original baseline value and a preset target baseline value; and
determining a plurality of actual offsets of the current baseline adjustment time window based on the target offset, and sequentially offsetting a baseline of the original human biological signal at a plurality of sampling points within the current baseline adjustment time window based on the plurality of actual offsets, so that the baseline of the original human biological signal within the current baseline adjustment time window gradually approaches the preset target baseline value;
wherein the plurality of actual offsets correspond to the baseline at the plurality of sampling points one by one.

9. The method according to claim 8, wherein the determining a plurality of actual offsets of the current baseline adjustment time window based on the target offset, and sequentially offsetting a baseline of the original human biological signal at a plurality of sampling points within the current baseline adjustment time window based on the plurality of actual offsets, comprises:
determining an initial baseline single-step variation based on the preset target baseline value and a preset initial actual offset before acquiring a baseline of the original human biological signal at a first sampling point within the current baseline adjustment time window;
determining a second actual offset based on the preset initial actual offset and the initial baseline single-step variation, and taking the second actual offset as a current actual offset;
offsetting the baseline at the first sampling point based on the current actual offset; and
determining a next baseline single-step variation based on the target offset and the current actual offset, determining a next actual offset based on the current actual offset and the next baseline single-step variation, offsetting the baseline at a next sampling point based on the next actual offset, then taking the next actual offset as the current actual offset, and repeating the step of determining a next baseline single-step variation based on the target offset and the current actual offset until at least one second preset condition is satisfied;
wherein the at least one second preset condition comprises:
the current actual offset belongs to a second target interval, wherein the second target interval is determined by the target offset; and
a window end time of the current baseline adjustment time window is reached.

10. The method according to claim 9, wherein when the current actual offset belongs to the second target interval and the window end time of the current baseline adjustment time window is not reached, the baseline of the original human biological signal at remaining sampling points within the current baseline adjustment time window is offset based on the current actual offset until the window end time of the current baseline adjustment time window is reached.

11. An apparatus for processing human biological signal data, wherein an original human biological signal is traversed with a plurality of preset peak-to-peak value adjustment time windows; and for the original human biological signal in any one of the plurality of preset peak-to-peak value adjustment time windows except a first preset peak-to-peak value adjustment time window, the any one of the plurality of preset peak-to-peak value adjustment time windows is taken as a current peak-to-peak value adjustment time window, and the apparatus comprises:
a first acquisition module configured to, when a previous peak-to-peak value adjustment time window of the current peak-to-peak value adjustment time window ends, acquire an original peak-to-peak value of the original human biological signal within the previous peak-to-peak value adjustment time window;
a first determination module configured to determine a target adjustment multiple of the current peak-to-peak value adjustment time window based on the original peak-to-peak value and a preset target peak-to-peak value; and
an adjustment module configured to determine a plurality of actual adjustment multiples of the current peak-to-peak value adjustment time window based on the target adjustment multiple, and sequentially adjust signal values of the original human biological signal at a plurality of sampling points within the current peak-to-peak value adjustment time window based on the plurality of actual adjustment multiples, so that a peak-to-peak value of the original human biological signal within the current peak-to-peak value adjustment time window gradually approaches the preset target peak-to-peak value; wherein the plurality of actual adjustment multiples correspond to the signal values at the plurality of sampling points one by one.

12. The apparatus according to claim 11, wherein the adjustment module is further configured to determine an initial multiple single-step variation based on the target adjustment multiple and a preset initial actual adjustment multiple before acquiring a signal value of the original human biological signal at a first sampling point within the current peak-to-peak value adjustment time window; determine a second actual adjustment multiple based on the preset initial actual adjustment multiple and the initial multiple single-step variation, and take the second actual adjustment multiple as a current actual adjustment multiple; adjust the signal value at the first sampling point based on the current actual adjustment multiple; determine a next multiple single-step variation based on the target adjustment multiple and the current actual adjustment multiple, determine a next actual adjustment multiple based on the current actual adjustment multiple and the next multiple single-step variation, adjust a signal value at a next sampling point based on the next actual adjustment multiple, then take the next actual adjustment multiple as the current actual adjustment multiple, and repeat the step of determining a next multiple single-step variation based on the target adjustment multiple and the current actual adjustment multiple until at least one first preset condition is satisfied; wherein the at least one first preset condition comprises: the current actual adjustment multiple belongs to a first target interval, wherein the first target interval is determined by the target adjustment multiple; and a window end time of the current peak-to-peak value adjustment time window is reached.

13. The apparatus according to claim 11, wherein an original human biological signal is traversed with a plurality of preset baseline adjustment time windows; and for the original human biological signal in any one of the plurality of preset baseline adjustment time windows except a first preset baseline adjustment time window, the any one of the plurality of preset baseline adjustment time windows is taken as a current baseline adjustment time window, and the apparatus further comprises:
a second acquisition module configured to, when a previous baseline adjustment time window of the current baseline adjustment time window ends, acquire an original baseline value of the original human biological signal within the previous baseline adjustment time window;
a second determination module configured to determine a target offset of the current baseline adjustment time window based on the original baseline value and a preset target baseline value; and
an offsetting module configured to determine a plurality of actual offsets of the current baseline adjustment time window based on the target offset, and sequentially offset a baseline of the original human biological signal at a plurality of sampling points within the current baseline adjustment time window based on the plurality of actual offsets, so that the baseline of the original human biological signal within the current baseline adjustment time window gradually approaches the preset target baseline value; wherein the plurality of actual offsets correspond to the baseline at the plurality of sampling points one by one.

14. The apparatus according to claim 13, wherein the offsetting module is further configured to determine an initial baseline single-step variation based on the preset target baseline value and a preset initial actual offset before acquiring a baseline of the original human biological signal at a first sampling point within the current baseline adjustment time window; determine a second actual offset based on the preset initial actual offset and the initial baseline single-step variation, and take the second actual offset as a current actual offset; offset the baseline at the first sampling point based on the current actual offset; determine a next baseline single-step variation based on the target offset and the current actual offset, determine a next actual offset based on the current actual offset and the next baseline single-step variation, offset the baseline at a next sampling point based on the next actual offset, then take the next actual offset as the current actual offset, and repeat the step of determining a next baseline single-step variation based on the target offset and the current actual offset until at least one second preset condition is satisfied; wherein the at least one second preset condition comprises: the current actual offset belongs to a second target interval, wherein the second target interval is determined by the target offset; and a window end time of the current baseline adjustment time window is reached.

15. An electronic device, comprising a memory and a processor; wherein the memory has stored thereon a computer program to be loaded by the processor to perform the method according to claim 1.

16. An electronic device, comprising a memory and a processor; wherein the memory has stored thereon a computer program to be loaded by the processor to perform the method according to claim 8.

17. A non-transitory computer-readable storage medium, wherein the non-transitory computer-readable storage medium has stored thereon a computer program to be loaded by a processor to perform the method according to claim 1.

18. A non-transitory computer-readable storage medium, wherein the non-transitory computer-readable storage medium has stored thereon a computer program to be loaded by a processor to perform the method according to claim 8.
